SEQUENCE LISTING (62 sequences)

SEQ ID NO: 1 - RNA, 7 nt, Artificial Sequence
  miR-CTLA4-1# seed Sequence
accuuca

SEQ ID NO: 2 - RNA, 7 nt, Artificial Sequence
  miR-CTLA4-2# seed Sequence
uucagug

SEQ ID NO: 3 - RNA, 7 nt, Artificial Sequence
  miR-CTLA4-3# seed Sequence
cugugcu

SEQ ID NO: 4 - RNA, 7 nt, Artificial Sequence
  miR-CTLA4-6# seed Sequence
uccaagg

SEQ ID NO: 5 - RNA, 21 nt, Artificial Sequence
  miR-CTLA4-1# + exo-motif
aaccuucaguggaguuggcga

SEQ ID NO: 6 - RNA, 21 nt, Artificial Sequence
  miR-CTLA4-2# + exo-motif
cuucaguggaguuggcgagca

SEQ ID NO: 7 - RNA, 21 nt, Artificial Sequence
  miR-CTLA4-3# + exo-motif
acugugcugcggaggacaaau

SEQ ID NO: 8 - RNA, 21 nt, Artificial Sequence
  miR-CTLA4-6# + exo-motif
auccaaggacugggagcuguu

SEQ ID NO: 9 - RNA, 7 nt, Artificial Sequence
  miR-CTLA4-4# seed Sequence
gacauuc

SEQ ID NO: 10 - RNA, 7 nt, Artificial Sequence
  miR-CTLA4-5# seed Sequence
aaccuca

SEQ ID NO: 11 - RNA, 7 nt, Artificial Sequence
  miR-CTLA4-7# seed Sequence
cucaugu

SEQ ID NO: 12 - RNA, 7 nt, Artificial Sequence
  miR-CTLA4-8# seed Sequence
gcaacgg

SEQ ID NO: 13 - RNA, 7 nt, Artificial Sequence
  miR-CTLA4-9# seed Sequence
ggcaacg

SEQ ID NO: 14 - RNA, 7 nt, Artificial Sequence
  miR-CTLA4-10# seed Sequence
gacugug

SEQ ID NO: 15 - RNA, 21 nt, Artificial Sequence
  miR-CTLA4-4# + exo-motif
cgacauucacggaggagaaua

SEQ ID NO: 16 - RNA, 21 nt, Artificial Sequence
  miR-CTLA4-5# + exo-motif
gaaccucacccuccaaggacu

SEQ ID NO: 17 - RNA, 21 nt, Artificial Sequence
  miR-CTLA4-7# + exo-motif
acucauguacccuccgccaua

SEQ ID NO: 18 - RNA, 21 nt, Artificial Sequence
  miR-CTLA4-8# + exo-motif
ggcaacgggaggcggaguuau

SEQ ID NO: 19 - RNA, 21 nt, Artificial Sequence
  miR-CTLA4-9# + exo-motif
gggcaacgggacggagauuua

SEQ ID NO: 20 - RNA, 21 nt, Artificial Sequence
  miR-CTLA4-10# + exo-motif
ugacugugcugcggcggacaa

SEQ ID NO: 21 - RNA, 4 nt, Artificial Sequence
  exo-motif
ggag

SEQ ID NO: 22 - RNA, 4 nt, Artificial Sequence
  exo-motif
ggac

SEQ ID NO: 23 - RNA, 4 nt, Artificial Sequence
  exo-motif
ggcg

SEQ ID NO: 24 - RNA, 4 nt, Artificial Sequence
  exo-motif
ggcc

SEQ ID NO: 25 - RNA, 4 nt, Artificial Sequence
  exo-motif
gggg

SEQ ID NO: 26 - RNA, 4 nt, Artificial Sequence
  exo-motif
gggc

SEQ ID NO: 27 - RNA, 4 nt, Artificial Sequence
  exo-motif
ugag

SEQ ID NO: 28 - RNA, 4 nt, Artificial Sequence
  exo-motif
ugac

SEQ ID NO: 29 - RNA, 4 nt, Artificial Sequence
  exo-motif
ugcg

SEQ ID NO: 30 - RNA, 4 nt, Artificial Sequence
  exo-motif
ugcc

SEQ ID NO: 31 - RNA, 4 nt, Artificial Sequence
  exo-motif
uggg

SEQ ID NO: 32 - RNA, 4 nt, Artificial Sequence
  exo-motif
uggc

SEQ ID NO: 33 - RNA, 4 nt, Artificial Sequence
  exo-motif
cgag

SEQ ID NO: 34 - RNA, 4 nt, Artificial Sequence
  exo-motif
cgac

SEQ ID NO: 35 - RNA, 4 nt, Artificial Sequence
  exo-motif
cgcg

SEQ ID NO: 36 - RNA, 4 nt, Artificial Sequence
  exo-motif
cgcc

SEQ ID NO: 37 - RNA, 4 nt, Artificial Sequence
  exo-motif
cggg

SEQ ID NO: 38 - RNA, 4 nt, Artificial Sequence
  exo-motif
cggc

SEQ ID NO: 39 - RNA, 4 nt, Artificial Sequence
  exo-motif
cccu

SEQ ID NO: 40 - RNA, 4 nt, Artificial Sequence
  exo-motif
cccg

SEQ ID NO: 41 - RNA, 4 nt, Artificial Sequence
  exo-motif
ccca

SEQ ID NO: 42 - RNA, 4 nt, Artificial Sequence
  exo-motif
uccu

SEQ ID NO: 43 - RNA, 4 nt, Artificial Sequence
  exo-motif
uccg

SEQ ID NO: 44 - RNA, 4 nt, Artificial Sequence
  exo-motif
ucca

SEQ ID NO: 45 - RNA, 4 nt, Artificial Sequence
  exo-motif
gccu

SEQ ID NO: 46 - DNA, 4 bp, Artificial Sequence
  exo-motif
gccg

SEQ ID NO: 47 - RNA, 4 nt, Artificial Sequence
  exo-motif
gcca

SEQ ID NO: 48 - RNA, 7 nt, Artificial Sequence
  exo-motif
ggaggac

SEQ ID NO: 49 - RNA, 10 nt, Artificial Sequence
  exo-motif
ggacugggag

SEQ ID NO: 50 - RNA, 7 nt, Artificial Sequence
  exo-motif
ggaggag

SEQ ID NO: 51 - RNA, 8 nt, Artificial Sequence
  exo-motif
ggacggag

SEQ ID NO: 52 - RNA, 10 nt, Artificial Sequence
  exo-motif
ggaggcggag

SEQ ID NO: 53 - DNA, 59 bp, Artificial Sequence
  sequences of miR-CTLA4-1#
aaccttcagtggagttggcgagttttggccactgactgactcgccaaccactgaaggtt

SEQ ID NO: 54 - DNA, 59 bp, Artificial Sequence
  sequences of miR-CTLA4-2#
cttcagtggagttggcgagcagttttggccactgactgactgctcgccctccactgaag

SEQ ID NO: 55 - DNA, 59 bp, Artificial Sequence
  sequences of miR-CTLA4-3#
actgtgctgcggaggacaaatgttttggccactgactgacatttgtcccgcagcacagt

SEQ ID NO: 56 - DNA, 59 bp, Artificial Sequence
  sequences of miR-CTLA4-4#
cgacattcacggaggagaatagttttggccactgactgactattctcccgtgaatgtcg

SEQ ID NO: 57 - DNA, 59 bp, Artificial Sequence
  sequences of miR-CTLA4-5#
gaacctcaccctccaaggactgttttggccactgactgacagtccttggggtgaggttc

SEQ ID NO: 58 - DNA, 59 bp, Artificial Sequence
  sequences of miR-CTLA4-6#
atccaaggactgggagctgttgttttggccactgactgacaacagctcagtccttggat

SEQ ID NO: 59 - DNA, 59 bp, Artificial Sequence
  sequences of miR-CTLA4-7#
actcatgtaccctccgccatagttttggccactgactgactatggcggggtacatgagt

SEQ ID NO: 60 - DNA, 59 bp, Artificial Sequence
  sequences of miR-CTLA4-8#
ggcaacgggaggcggagttatgttttggccactgactgacataactccctcccgttgcc

SEQ ID NO: 61 - DNA, 59 bp, Artificial Sequence
  sequences of miR-CTLA4-9#
gggcaacgggacggagatttagttttggccactgactgactaaatctctcccgttgccc

SEQ ID NO: 62 - DNA, 59 bp, Artificial Sequence
  sequences of miR-CTLA4-10#
tgactgtgctgcggcggacaagttttggccactgactgacttgtccgccagcacagtca

(19) United States
(12) Patent Application Publication
(10) Pub. No.: US 2022/0347243 A1
(43) Pub. Date: Nov. 3, 2022

(54) PHARMACEUTICAL COMPOSITIONS, KITS AND METHODS FOR TREATING TUMORS

(71) Applicant: IMMVIRA CO., LIMITED, Shenzhen (CH)

(72) Inventors: Xiaoqing Chen, Shenzhen (CN); Xusha Zhou, Shenzhen (CN); Bernard Roizman, Shenzhen (CN); Grace Guoying Zhou, Shenzhen (CN)

(21) Appl. No.: 17/622,217

(22) PCT Filed: Jul. 4, 2019
(86) PCT No.: PCT/CN2019/094645

§ 371 Date: Dec. 22, 2021

(51) Int. Cl. A61K 35/763; A61P 35/00; C07K 16/28; A61K 38/20; C12N 7/00; C12N 15/86; C12N 15/113

(52) U.S. Cl. CPC A61K 35/763; A61P 35/00; C07K 16/2818; A61K 38/208; C12N 7/00; C12N 15/86; C12N 15/1135; C12N 2710/16642; C12N 2710/16632; C12N 2310/141

(57) ABSTRACT

Provided is a composition for treating tumors in a subject comprising a therapeutically effective amount of an exosome carrying CTLA4-targeting miRNA and a therapeutically effective amount of an oncolytic herpes simplex virus expressing an immunostimulatory agent or both an immunostimulatory agent and an anti-PD-1 antibody. Wherein an exo-motif operably links to the seed sequence of the CTLA4-targeting miRNA to enhance the packaging of the CTLA4-targeting miRNA into the exosome.

### TECHNICAL FIELD

The present invention is related to a pharmaceutical composition for treating a tumor, and in particular, to a pharmaceutical composition comprising a therapeutically effective amount of an exo some carrying CTLA-4 targeting miRNA and a therapeutically effective amount of an oncolytic herpes simplex virus (oHSV). The present invention is also related to a kit comprising the exo some carrying CTLA-4 targeting miRNA and an oHSV, and methods of using the pharmaceutical composition and the kit for treating a tumor.

### BACKGROUND

Cancer as a disease is a multifaceted foe which may succumb to the prescribed treatment and may develop resistance against various therapies. A subset of cells within tumors are resistant to conventional treatment modalities and may be responsible for disease recurrence.

Surgical treatment of cancer is a common local treatment. In addition to some malignant tumors of the blood system, such as leukemia, lymphoma, etc., other various malignant tumors have one or more tangible solid tumors, which can be surgically removed. However, surgery always has certain risks and often has other comorbidities or potential organ dysfunction.

Non-surgical treatments of cancer (mainly conventional chemotherapy, targeted biological therapies, and radiotherapy) have not generated completely satisfactory results to date. The ongoing problems include low target selectivity, drug resistance, inability to effectively address metastatic disease and severe side effects. In contrast, immunotherapies that overall provoke host immunity to induce a systemic response against tumors currently offer much clinical promise.

Oncolytic herpes simplex viruses (oHSV) are being extensively investigated for treatment of solid tumors. As a group, they pose many advantages over traditional cancer therapies. Specifically, oHSV usually embody a mutation that makes them susceptible to inhibition by some aspect of innate immunity. As a consequence, they replicate in cancer cells in which one or more innate immune responses to infection are compromised but not in normal cells in which the innate immune responses are intact. oHSV are usually delivered directly into the tumor mass in which the virus can replicate. Because it is delivered to the target tissue rather than systemically, there are no side effect characteristics of anti-cancer drugs. Viruses characteristically induce adaptive immune responses that curtail their ability to be administered multiple times. oHSV has been administered to tumors multiple times without evidence of loss of potency or induction of adverse reaction such as inflammatory responses. HSV are large DNA viruses capable of incorporating into their genomes foreign DNA and to regulate the expression of these gene on administration to tumors. The foreign genes suitable for use with oHSV are those that help to induce an adaptive immune response to the tumor.

The defect in overcoming the cellular innate immune response determines the range of tumors in which the virus exhibits its oncolytic oHSV as an anti-cancer agent. The more extensive the deletions the more restrictive is the range of cancer cells in which the oHSV is effective depends on the function of the deleted viral gene. Most recent oHSV incorporate at least one cellular gene to bolster its anti-cancer activity.

The success of the oHSV based therapy hinges on the extent of destruction of cancer cells. Early in the development of oHSV it was recognized that that HSV alone could not kill all cancer cells in a solid tumor and that it is unlikely that oHSV treatment could effectively eliminate all cancer cells and that destruction of tumors by oHSV in clinical trials had to involve an adaptive immune response to the tumor. Further studies have shown that the antitumor immune response generated by the infected tumor cell debris could be augmented by incorporation of cytokines. Comparison of oHSV bereft of cytokine gene with oHSV incorporating an immunostimulatory cytokine confirmed this hypothesis and led ultimately to the incorporation of GM-CSF into oHSV developed for treatment of melanoma.

Incorporation of genes encoding immunostimulatory cytokines enhances the immune response to the tumor but does no effectively enhance the cytoxicity caused by T cells that is critical for anti-tumor effects. Tumors co-opt PD-1 and CTLA-4 inhibitory pathways to silence the immune system. PD-1 expresses on activated T cells and other hematopoietic cells while CTLA-4 expresses on activated T cells including regulatory T cells. Tumors employ PD-1 and CTLA-4 inhibitory pathway to evade the host immune response.

Although extended studying and testing in pre-clinical and clinical setting, an unmet need continues to exist for methods of treating tumors.

### SUMMARY

In one aspect, the invention is related to a pharmaceutical composition comprising a therapeutically effective amount of an exosome carrying a miRNA targeting CTLA-4 and a therapeutically effective amount of an oncolytic herpes simplex virus (oHSV) expressing an immuno stimulatory agent or immuno stimulatory agent and anti-PD-1 antibody.

The exosome comprises an exosome-packaging-associated motif (also referred to as “exo-motif” hereinafter) operably linked, optionally through a linker, to the miRNA targeting CTLA-4. In one embodiment, the exosome comprises an inhibitory amount of CTLA4-targeting miRNA, wherein the CTLA-4 targeting miRNA has a seed sequence binding to mRNA of CTLA-4; and an exo-motif operably linked to the seed sequence of the CTLA-4 targeting miRNA to enhance the packaging of the CTLA-4 targeting miRNA into the exosome. In some embodiments, the exo-motif is located downstream and covalently linked to the seed sequence of the CTLA-4 targeting miRNA. In some embodiments, the exo-motif is located downstream and linked to the seed sequence of the CTLA-4 by a linker. In some embodiments, the exo-motif is obtained by mutation of one or more nucleic acids of the CTLA-4 targeting miRNA except for the seed sequence. In some embodiments, the exo-motif is a two-fold motif generated through combination of two single exo-motifs. In some embodiments, the CTLA-4 targeting miRNA and the exo-motif, when operably linked, share at least one or two nucleotides.

The oHSV is recombinant oncolytic HSV-1 expressing an immunostimulatory agent or both an immunostimulatory agent and an anti-PD-1 antibody.

Another aspect of the invention is related to a pharmaceutical composition comprising a therapeutically effective amount of an exosome carrying a miRNA targeting CTLA-4, a therapeutically effective amount of an oHSV, and a pharmaceutically acceptable carrier. The exosome comprises an exosome-packaging-associated motif operably linked, optionally through a linker, to the miRNA targeting CTLA-4. The oHSV expressing an immunostimulatory agent or both an immunostimulatory agent and an anti-PD-1 antibody.

Another aspect of the invention is related to a kit comprising an exo some carrying a miRNA targeting CTLA-4 and an oHSV expressing an immunostimulatory agent or both an immunostimulatory agent and an anti-PD-1 antibody for treating a tumor. The kit may further comprise instructions for using the exo some and the oHSV for treating tumors.

A further aspect of the invention is related to a method for treating tumor in a subject, comprising concurrently administering to the subject a therapeutically effective amount of the exo some carrying a miRNA targeting CTLA4 and therapeutically effective amount of an oHSV expressing an immunostimulatory agent or both an immunostimulatory agent and an anti-PD-1 antibody.

A further aspect of the invention is related to a method for enhancing efficacy of an oHSV therapy in a subject comprising administering to the subject in need thereof a therapeutically effective amount of an exosomes carrying miRNA targeting CTLA4 of the invention in addition to the oHSV therapy.

Other aspects of the invention will be readily available from reading the description below.

### BRIEF DESCRIPTION OF DRAWINGS

FIG. 1. Panel A is schematic diagram of the plasmid encoding miRNAs targeting CTLA4. The plasmid consisted of the sequence encoding EGFP containing in its 3′-UTR the sequence encoding the designed miRNAs targeting CTLA-4 gene (miR-CTLA-4). Panel B shows the nucleotide sequence of miRNAs targeting mouse CTLA-4 gene. The nucleotides highlighted in bold, italic, and underline indicate exosome-packaging-associated motifs (EXO-motifs). Panel C shows down-regulation of CTLA4 by designed miRNAs. HEp-2 cells seeded in 24-well plates were co-transfected with 0.25 μg of plasmids expressing 10 DNA sequences encoding the miRNAs against CTLA-4 (1#-10#) or non-target miRNA (NT) and 0.25 μg of plasmid encoding a his-tagged mouse CTLA-4 (His-tagged CTLA-4). The cells were harvested after 72 h post transfection. Accumulated of CTLA4 and GAPDH were measured as known to those skilled in the art.

FIG. 2. Characterization of exosome carrying miR-CTLA-4. HEp-2 cells seeded in T150 flask were transfected with 10 μg of the miR-CTLA-4-3# plasmid or plasmid expresses non-target miRNA (NT) then incubated in serum free medium. After 48 h the medium was collected and the exosomes were purified as described in Materials and Methods. The purified exosomes were subjected to 2 series of analyses. First (Panel A) equal amounts of cells in which the exosomes were produced and equal amounts of exosomes were solubilized, subjected to electrophoresis in a denaturing gel were probed with antibodies to CD9, Flotilin-1 and Calnexin. Typically, the purified exosomes contained CD9, Flotilin-1 but lacked Calnexin. The size distributions of exosomes (Panel B) produced by transfected cells were done as described in in Materials and Methods.

FIG. 3. The impact of exosomes carrying miR-CTLA-4 administered alone (Panel A) or concurrently T1012G (Panel B), T2850 (Panel C) or T3855 (panel D) on MFC tumor growth. MFC tumor cells were injected subcutaneously in the right flanks of C57BL/6J mice. MFC tumors averaging 80 mm³ were injected in groups of 8 animals intratumorally with 10 μg of exosome alone or concurrently with 50 μl of 1×10⁷ pfu of T1012G, T2850 or T3855. All of the studies were done concurrently but the results are shown in 4 panels. Tumor volumes are shown as mean±SEM of 8 animals in each group.

### DETAILED DESCRIPTION OF THE INVENTION

### Definitions

It is to be noted that the term “a” or “an” entity refers to one or more of that entity; for example, “an exosome,” is understood to represent one or more exosomes. As such, the terms “a” (or “an”), “one or more,” and “at least one” can be used interchangeably herein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

The term “linker” as used herein refers to a short fragment of nucleotide sequence containing two or more nucleotides which may be same or different, wherein the nucleotides are selected from a group consisting of Adenine (A), Guanine (G), Cytosine (C), Thymine (T) and Uracil (U).

As used herein, the terms “treat” or “treatment” refer to both therapeutic treatment and prophylactic or preventative measures, wherein the object is to prevent or slow down (lessen) an undesired physiological change or disorder, such as the progression of tumor. Beneficial or desired clinical results include, but are not limited to, alleviation of symptoms, diminishment of extent of disease, stabilized (i.e., not worsening) state of tumor, inhibition of tumor growth, reducing the volume of the tumor, delay or slowing of tumor progression, amelioration or palliation of the tumor state, and remission (whether partial or total), whether detectable or undetectable. Those in need of treatment include those already have a tumor as well as those who are prone to have a tumor.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a patient in need of treatment” or “a subject in need of treatment” includes subjects, such as mammalian subjects, that would benefit from administration of a composition of the present disclosure used, e.g., for detection, for a diagnostic procedure and/or for treatment.

The present invention employs, among others, antisense oligomer and similar species for use in modulating the function or effect of nucleic acid molecules encoding CTLA4. The hybridization of an oligomer of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of CTLA4. In the context of the present invention, “modulation” and “modulation of expression” mean decrease (inhibition) in the amount or levels of a nucleic acid molecule encoding the gene, e.g., DNA or RNA. mRNA is often a preferred target nucleic acid.

In the context of this invention, “hybridization” means the pairing of complementary strands of oligomers. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomers and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense oligomer need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense oligomer are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomer which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art.

In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

As used herein, the term “microRNA”, “miRNA”, or “miR” refers to RNAs that function post-transcriptionally to regulate expression of genes, usually by binding to complementary sequences in the three prime (3′) untranslated regions (3′ UTRs) of target messenger RNA (mRNA) transcripts, usually resulting in gene silencing. miRNAs are typically small regulatory RNA molecules, for example, 21 or 22 nucleotides long. The terms “microRNA”, “miRNA”, and “miR” are used interchangeably.

As used herein, the term “tumor” refers to a malignant tissue comprising transformed cells that grow uncontrollably (i.e., is a hyperproliferative disease). Tumors include leukemias, lymphomas, myelomas, plasmacytomas, and the like; and solid tumors. Examples of solid tumors that can be treated according to the invention include but are not limited to sarcomas and carcinomas such as melanoma, fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

The term “CTLA4” as used herein refers to “cytotoxic T-lymphocyte-associated protein 4” which is one of many coinhibitory molecules that can attenuate T cell activation by inhibiting co-stimulation and transmitting inhibitory signals to T cells. Amino acid sequences of CTLA4 are available from NCBI through accession numbers NP_033973.2 or NP_001268905.1. CTLA4 is also known as Ctla-4, Cd152 or Ly-56. The NCBI sequence accession numbers of CTLA4 is NC_000067.6 and gene ID is 12477. The human CTLA4 gene encodes a 233 amino-acid protein belonging to the immunoglobulin superfamily. CTLA4 consists of one V-like domain flanked by two hydrophobic regions. CTLA4 also can change the structure of immune synapses, which serve a pivotal role in T cell proliferation and differentiation CTLA4. Polymorphisms in CTLA4 have been associated with susceptibility to multiple diseases, including type I diabetes, primary biliary cirrhosis and Graves' disease.

The term “IL-12” as used herein refers to “interleukin 12” which is a cytokine with potent antitumor effects. Thus IL-12 induces a TH-1 type immune response, which may provide a durable antitumor effect. IL-12 has been reported to have in vivo anti-angiogenic activity, which may also contribute to its antitumor effects. Lastly IL-12 has been reported to stimulate the production of high levels of IFN-γ, which has multiple immunoregulatory effects including the capacity to stimulate the activation of CTLs, natural killer cells, and macrophages and to induce/enhance the expression of class II MHC antigens. IFN-γ plays a significant role in the process of inducing T-cell migration to tumor sites. Increases in the intratumoral levels of IFN-γ correlated with a decrease in the size of the tumor burden.

Programmed Cell Death 1 (PD-1) is a 50-55 kDa type I transmembrane receptor originally identified by subtractive hybridization of a mouse T cell line undergoing apoptosis (Ishida et al., 1992, Embo J. 11:3887-95). A member of the CD28 gene family, PD-1 is expressed on activated T, B, and myeloid lineage cells (Greenwald et al., 2005.Annu. Rev. Immunol. 23:515-48; Sharpe et al., 2007, Nat. Immunol. 8:239-45). Human and murine PD-1 share about 60% amino acid identity with conservation of four potential N-glycosylation sites and residues that define the lg-V domain. PD-1 negatively modulates T cell activation, and this inhibitory function is linked to an immunoreceptor tyrosine-based inhibitory motif (Irm) of its cytoplasmic domain (Parry et al., 2005, Mol. Cell. Biol. 25:9543-53). Disruption of this inhibitory function of PD-1 can lead to autoimmunity.

As used herein, an “antibody” or “antigen-binding polypeptide” refers to a polypeptide or a polypeptide complex that specifically recognizes and binds to one or more antigens. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody also encompasses polypeptides or polypeptide complexes that, upon activation possess antigen-binding capabilities.

By “therapeutically effective amount” it is meant that the oncolytic virus and/or the exosome of the present disclosure is administered in an amount that is sufficient for “treatment” as described above. The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

miRNAs Targeting CTLA4

The terms “miRNAs targeting CTLA4”, “a miRNA targeting CTLA4”, and “CTLA4-targeting miRNA” which are used interchangeably herein, refer to a small non-coding RNA (microRNA or miRNA) designed to target or specifically bind to mRNA encoding protein CTLA4 such that the transcription, translation and, in turn, expression of the CTLA4 in a cell is impaired, reduced, or eliminated. As described above, miRNA is not necessarily bind to target mRNA by 100% specificity. It is known that miRNA has a seed sequence (2-8 nucleotides from 5′ end) which determines the specificity of biding to a target mRNA, while the remaining nucleotides are not necessarily exactly complementary to the target mRNA. Therefore, in one embodiment, the miRNA has a seed sequence of any of nucleotide sequences SEQ ID NO. 1, SEQ ID NO: 2, SEQ ID NO. 3 or SEQ ID NO. 4. In some embodiments, the miRNA targeting CTLA4 blocks the expression of CTLA4 protein in a cell after delivered to a tumor cell.

Exosomes Carrying miRNA Targeting CTLA4

Exosomes are small, relatively uniform-sized vesicles derived from cellular membranes. For example, exosomes may have a diameter of about 30 to about 100 nm. They contain several key proteins (e.g. CD9, CD63, CD81, CD82, Annexin, Flotillin, etc) and in addition they package proteins, mRNAs, long non-coding RNAs and miRNAs. Exosomes transport the payload from cell to cell. On entry into recipient cells the exosome payload is released into cytoplasm.

In some embodiments, the miRNA targeting CTLA4 is delivered to a cell via an exosome. Therefore, in one embodiment, an exosome carrying any of the CTLA4-targeting miRNAs as described above is provided. The present invention uses a fragment of nucleotide sequence, referred to as “exo-motif” herein, to facilitate or enhance the packaging of a miRNA into an exo some. In one embodiment, the exo-motif is selected from any of the sequences identified in Table 1.

TABLE 1
Sequences of exo-motifs used with
miRNAs of the invention

| Sequence | Nucleotide | Sequence | Nucleotide |
|---|---|---|---|
| ID | Sequence | ID | Sequence |
| SEQ ID | 5′-GGAG-3′ | SEQ ID | 5′-CGCC-3′ |
| NO. 21 | | NO. 36 | |
| SEQ ID | 5′-GGAC-3′ | SEQ ID | 5′-CGGG-3′ |
| NO. 22 | | NO. 37 | |
| SEQ ID | 5′-GGCG-3′ | SEQ ID | 5′-CGGC-3′ |
| NO. 23 | | NO. 38 | |
| SEQ ID | 5′-GGCC-3′ | SEQ ID | 5′-CCCU-3′ |
| NO. 24 | | NO. 39 | |
| SEQ ID | 5′-GGGG-3′ | SEQ ID | 5′-CCCG-3′ |
| NO. 25 | | NO. 40 | |
| SEQ ID | 5′-GGGC-3′ | SEQ ID | 5′-CCCA-3′ |
| NO. 26 | | NO. 41 | |
| SEQ ID | 5′-UGAG-3′ | SEQ ID | 5′-UCCU-3′ |
| NO. 27 | | NO. 42 | |
| SEQ ID | 5′-UGAC | SEQ ID | 5′-UCCG-3′ |
| NO. 28 | | NO. 43 | |
| SEQ ID | 5′-UGCG | SEQ ID | 5′-UCCA-3′ |
| NO. 29 | | NO. 44 | |
| SEQ ID | 5′-UGCC | SEQ ID | 5′-GCCU-3′ |
| NO. 30 | | NO. 45 | |
| SEQ ID | 5′-UGGG | SEQ ID | 5′-GCCG-3′ |
| NO. 31 | | NO. 46 | |
| SEQ ID | 5′-UGGC | SEQ ID | 5′-GCCA-3′ |
| NO. 32 | | NO. 47 | |
| SEQ ID | 5′-CGAG | SEQ ID | 5′-GGAGGAC-3′ |
| NO. 33 | | NO. 48 | |
| SEQ ID | 5′-CGAC | SEQ ID | 5′-GGACUGGGAG-3′ |
| NO. 34 | | NO. 49 | |
| SEQ ID | 5′-CGCG-3′ | SEQ ID | 5′-GGAGGAG-3′ |
| NO. 35 | | NO. 50 | |
| SEQ ID | 5′-GGACGGAG-3′ | SEQ ID | 5′-GGAGGCGGAG-3′ |
| NO. 51 | | NO. 52 | |

In some embodiments, the exo-motifs are used in combination. For example, two or more exo-motifs as identified in the Table are combined to form a two-fold exo-motif. The motifs can be combined linearly by linking the 5′-end of one exo-motif to the 3′-end of another exo-motif. In this context, when the first nucleotide of the 5′-end of one exo-motif is identical with the last nucleotide of the 3′-end of another exo-motif, one of the identical nucleotides can be designed to be omitted. For example, “GGAG” (SEQ ID NO. 21) is combined with “GGAC” (SEQ ID NO. 22) to form a two-fold exo-motif “GGAGGAC” (SEQ ID NO. 48). When the first nucleotide of the 5′-end of one exo-motif is different from the last nucleotide of the 3′-end of another exo-motif, the two exo-motifs can be connected by a linker or directly by a covalent bond. For example, “GGAC” (SEQ ID NO.22) may be combined with “GGAG” (SEQ ID NO.21) by a linker “TG” to form a two-fold exo-motif “GGACUGGGAG” (SEQ ID NO. 49), “GGAC” (SEQ ID NO.22) may also be combined with “GGAG” (SEQ ID NO.21) by a covalent bond to form a two-fold exo-motif “GGACGGAG” (SEQ ID NO. 51). The present invention also contemplates a three-fold or more exo-motif, i.e., an exo-motif consisted of three or more motifs of SEQ ID NO. 21 to SEQ ID NO. 47. Therefore, the term “exo-motif” used herein is meant to include nucleotide sequences that are able to enhance or facilitate packaging of miRNA to an exosome, including any of the single exo-motif of SEQ ID NO. 21 to SEQ ID NO. 47 and any two-fold (e.g. any one of SEQ ID NO. 48-52), three-fold or more fold exo-motifs generated by the combinations of the single motifs.

In the present invention, the exo-motif is operably linked to the seed sequence of the miRNA. The term “operably linked” refers to functional linkage between a regulatory sequence (e.g. the exo-motif) and a nucleic acid sequence (e.g., the seed sequence of the miRNA) resulting in an enhance of, or facilitating the packaging of the miRNA into an exosome. For example, a first nucleic acid sequence is operably linked with a second nucleic acid sequence when the first nucleic acid sequence is placed in a functional relationship with the second nucleic acid sequence. Operably linked RNA sequences can be contiguous with each other or can be connected with a linker.

In some embodiments, an exo-motif is located downstream the seed sequence of the miRNA. In some embodiments, an exo-motif is located upstream the seed sequence of the miRNA. In some embodiments, the seed sequence of the miRNA is flanked by exo-motifs. In one embodiment, an exo-motif is operably linked to the seed sequence of the miRNA. In one embodiment, an exo-motif is obtained by mutation of one or more of the nucleotide sequences of the miRNA except for the seed sequence. In one embodiment, the miRNA targeting CTLA4 with exo-motif contains a nucleotide sequence of SEQ ID NO. 5, SEQ ID NO. 6, SEQ ID NO. 7 or SEQ ID NO. 8. In one embodiment, the miRNA targeting CTLA4 with exo-motif is a nucleotide sequence of SEQ ID NO. 5, SEQ ID NO. 6, SEQ ID NO. 7 or SEQ ID NO. 8.

In some embodiments where an exo-motif is located downstream the seed sequence of the miRNA, the 3′ end last nucleotide of the seed sequence and the 5′ end first nucleotide of the exo-motif share a same nucleotide, for example, guanine nucleotide “G”. For example, SEQ ID NO. 6 shows the sharing of the guanine nucleotide “G” between the exo-motif and the seed sequence. In some embodiments where an exo-motif is located downstream the seed sequence of the miRNA, the 3′ end last two nucleotides of the seed sequence and the 5′ end first two nucleotides of the exo-motif share the same two nucleotides, for example, two guanine nucleotides “GG”. For example, SEQ ID NO. 8 shows the sharing of the two guanine nucleotides “GG” between the exo-motif and the seed sequence.

In some embodiments, the exo-motif is located downstream of the seed sequence of the miRNA and is connected to the seed sequence of the miRNA by a linker, for example, “GC”. For example, SEQ ID NO. 7 shows the exo-motif and the seed sequence are connected by a linker “GC”.

In addition to the seed sequence and the exo-motif, the miRNA also includes additional nucleic acid sequence to facilitate binding to the target region of the mRNA. These additional nucleic acids are normally located downstream the exo-motif with a length of several nucleotides, e.g., 1 to 10 nucleotides. The additional nucleic acid sequences are preferably complementary to the corresponding segment of the target mRNA, but, as described above, not necessarily 100% complementary.

Methods for transferring miRNAs into an exosome are available in the art, such as by co-transfecting a cell with a miRNA expression vector and a plasmid encoding CTLA4, as described in the Example. Isolation, identification or characterization of an exosome is technically feasible in the art. Several proteins, e.g. CD9, CD63, CD81, CD82, Annexin, Flotillin, etc can be used as a marker of exosomes. Other methods for packaging miRNAs into exosomes may also be applicable with the present invention.

The exosome of the present invention contains an inhibitory amount of miRNA targeting CTLA4. An inhibitory amount is meant an amount sufficient for inhibiting the expression of the protein CTLA4 once the miRNA in question was delivered into a tumor cell.

The table below lists the nucleic acid sequences of miRNAs, seed sequences, and miRNA-motif used in the Example of the invention.

TABLE 2
Nucleic acid sequences of miRNAs, seed
sequences, and miRNAs linked with
exo-motifs

| Sequence ID | Identity | Description | Nucleic Acid Sequence |
|---|---|---|---|
| SEQ ID NO. 1 | miR-CTLA4-1# | Seed Sequence | 5′-ACCUUCA-3′ |
| SEQ ID NO. 2 | miR-CTLA4-2# | Seed Sequence | 5′-UUCAGUG-3′ |
| SEQ ID NO. 3 | miR-CTLA4-3# | Seed Sequence | 5′-CUGUGCU-3′ |
| SEQ ID NO. 4 | miR-CTLA4-6# | Seed Sequence | 5′-UCCAAGG-3′ |
| SEQ ID NO. 5 | miR-CTLA4-1# | miRNA + exo-motif | 5′-AACCUUCAGUGGAGUUGGCGA-3′ |
| SEQ ID NO. 6 | miR-CTLA4-2# | miRNA + exo-motif | 5′-CUUCAGUGGAGUUGGCGAGCA-3′ |
| SEQ ID NO. 7 | miR-CTLA4-3# | miRNA + exo-motif | 5′-ACUGUGCUGCGGAGGACAAAU-3′ |
| SEQ ID NO. 8 | miR-CTLA4-6# | miRNA + exo-motif | 5′-AUCCAAGGACUGGGAGCUGUU-3′ |
| SEQ ID NO. 9 | miR-CTLA4-4# | Seed Sequence | 5′-GACAUUC-3′ |
| SEQ ID NO. 10 | miR-CTLA4-5# | Seed Sequence | 5′-AACCUCA-3′ |
| SEQ ID NO. 11 | miR-CTLA4-7# | Seed Sequence | 5′-CUCAUGU-3′ |
| SEQ ID NO. 12 | miR-CTLA4-8# | Seed Sequence | 5′-GCAACGG-3′ |
| SEQ ID NO. 13 | miR-CTLA4-9# | Seed Sequence | 5′-GGCAACG-3′ |
| SEQ ID NO. 14 | miR-CTLA4-10# | Seed Sequence | 5′-GACUGUG-3′ |
| SEQ ID NO. 15 | miR-CTLA4-4# | miRNA + exo-motif | 5′-CGACAUUCACGGAGGAGAAUA-3′ |
| SEQ ID NO. 16 | miR-CTLA4-5# | miRNA + exo-motif | 5′-GAACCUCACCCUCCAAGGACU-3′ |
| SEQ ID NO. 17 | miR-CTLA4-7# | miRNA + exo-motif | 5′-ACUCAUGUACCCUCCGCCAUA-3′ |
| SEQ ID NO. 18 | miR-CTLA4-8# | miRNA + exo-motif | 5′-GGCAACGGGAGGCGGAGUUAU-3′ |
| SEQ ID NO. 19 | miR-CTLA4-9# | miRNA + exo-motif | 5′-GGGCAACGGGACGGAGAUUUA-3′ |
| SEQ ID NO. 20 | miR-CTLA4-10# | miRNA + exo-motif | 5′-UGACUGUGCUGCGGCGGACAA-3′ |

Oncolytic Herpes Simplex Virus (oHSV)

The oncolytic herpes simplex virus (oHSV) as used herein refers to any oncolytic type 1 herpes simplex viruses (HSV-1) known in the art designed, usable or effective to destruct a tumor cell. In addition, the oHSV used in the present disclosure can also be genetically engineered, so that one or more of the features of the natural oHSV is deleted. In addition or alternatively, a naturally occurring oHSV may be genetically engineered to introduce to the genome of the virus one or more exogenous fragments of coding sequences, so as to provide one or more additional functionality of the virus, such as immunotherapeutic or immunostimulatory properties.

It will be appreciated by a skilled person in the art that the exact starting and ending positions of the nucleotides to be deleted according to the present disclosure depend on the strains and genome isomers of the HSV-1 virus and can be easily determined by known techniques in the art. In some embodiments, the deletion causes the excision of nucleotides 117005 to 132096 in the genome. In some embodiments, the oHSV is selected from the strain 17 (GenBank Accession No. NC 001806.2) the strain KOS 1.1 (GenBank Accession No. KT899744) or the strain F (GenBank Accession No. GU734771.1) of the HSV-10 In some embodiments, the oHSV is the strain F of the HSV-1.

In some embodiments, the oHSV is a genetically engineered HSV-1 F strain expressing an immunostimulatory agent which is selected from GM-CSF, IL-2, IL-5, IL-12, IL-15, IL-24 and IL-27. In some embodiments, the oHSV is a genetically engineered HSV-1 F strain expressing IL-12 alone. In some embodiments, the oHSV is a genetically engineered HSV-1 F strain expressing both IL-12 and anti-PD-1 antibody.

### Methods and Therapies

An aspect of the disclosure provides a method for treatment of tumor in a subject comprising administering to the subject in need thereof a therapeutically effective amount of an exosome carrying miRNA targeting CTLA4 and a therapeutically effective amount of an oHSV expressing an immunostimulatory agent or both an immunostimulatory agent and an anti-pd-1 antibody.

An aspect of the disclosure provides a method for enhancing efficacy of an oHSV therapy in a subject comprising administering to the subject in need thereof a therapeutically effective amount of an exo some carrying miRNA targeting CTLA4 of the invention in addition to the oHSV therapy. In some embodiments, the oHSV expressing an immunostimulatory agent or both an immunostimulatory agent and an anti-PD-1 antibody. In some embodiments, the oHSV expresses IL-12 alone. In some embodiments, the oHSV expresses IL-12 and anti PD-1 antibody.

In some embodiments, in the methods of the disclosure, the administering of the exosomes carrying miRNA targeting CTLA4 inhibitor and the oHSV expressing an immunostimulatory agent or both an immunostimulatory agent and an anti-PD-1 antibody is carried out by administering to the subject a pharmaceutical composition comprising a therapeutically effective amount of the exosome carrying miRNA targeting CTLA4 and a therapeutically effective amount of an oHSV expressing an immunostimulatory agent or both an immuno stimulatory agent and an anti-PD-1 antibody, and a pharmaceutically acceptable carrier.

In some embodiments, in the methods of the disclosure, the administering of the exosomes carrying miRNA targeting CTLA4 and the oHSV expressing an immunostimulatory agent or both an immunostimulatory agent and an anti-PD-1 antibody is carried out by administering to the subject the exosomes carrying miRNA targeting CTLA4 and the oHSV expressing an immunostimulatory agent or both an immunostimulatory agent and an anti-PD-1 antibody separately. In some embodiments, the exosomes carrying miRNA targeting CTLA4 is administered before, simultaneously or after the administering of the oHSV expressing an immunostimulatory agent or both an immunostimulatory agent and an anti-PD-1 antibody. In such instances, it is contemplated that one may administer the subject with both modalities within about 12 to 72 hrs of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In some embodiments, the exosomes carrying miRNA targeting CTLA4 is administered simultaneously with the administering of the oHSV expressing an immunostimulatory agent or both an immunostimulatory agent and an anti-PD-1 antibody. In some embodiments, the exosomes carrying miRNA targeting CTLA4 is administered in the form of a pharmaceutical composition comprising a therapeutically effective amount of the exosomes carrying miRNA targeting CTLA4 and a pharmaceutically acceptable carrier, and the oHSV expressing an immunostimulatory agent or both an immunostimulatory agent and an anti-PD-1 antibody is administered in the form of a pharmaceutical composition comprising a therapeutically effective amount of the oHSV expressing an immunostimulatory agent or both an immunostimulatory agent and an anti-PD-1 antibody and a pharmaceutically acceptable carrier. In such embodiments, the pharmaceutical composition comprising a therapeutically effective amount of the exosomes carrying miRNA targeting CTLA4 and a pharmaceutically acceptable carrier, and the pharmaceutical composition comprising a therapeutically effective amount of the oHSV expressing an immunostimulatory agent or both an immunostimulatory agent and an anti-PD-1 antibody and a pharmaceutically acceptable carrier may be packaged in a single kit.

In certain embodiments, any of the pharmaceutical composition is administered parenterally or non-parenterally, e.g. intratumorally, intravenously, intramuscularly, percutaneously or intracutaneously. In some embodiments, any of the pharmaceutical composition is preferably administered intratumorally.

In certain embodiments, the method of treating a tumor is to enhance the anti-tumor efficacy of oHSV therapy, for example, in terms of inhibiting tumor growth, and/or reducing the volume of tumors. Thus, in some embodiments, the disclosure provides a method for treating a tumor comprising administering a therapeutically effective amount of the exo some in combination with a therapeutically effective amount of the oHSV as described above to a subject in need thereof. In certain embodiments, the methods of treating a tumor prevent the onset, progression and/or recurrence of a symptom associated with a tumor. Thus, in some embodiments, a method for preventing a symptom associated with a tumor in a subject comprises administering a therapeutically effective amount of the exosome and a therapeutically effective amount of the oHSV as described above to a subject in need thereof.

In some embodiments, the oHSV is a genetically engineered HSV-1 F strain expressing an immuno stimulatory agent which is selected from GM-CSF, IL-2, IL-5, IL-12, IL-15, IL-24 and IL-27. In some embodiments, the oHSV is a genetically engineered HSV-1 F strain expressing IL-12 alone. In some embodiments, the oHSV is a genetically engineered HSV-1 F strain expressing both IL-12 and anti-PD-1 antibody.

The methods of the disclosure are contemplated to treat various tumors, especially solid tumors. Examples of solid tumors that can be treated according to the invention include but are not limited to sarcomas and carcinomas such as melanoma, fibrosarcoma, myosarcoma, liposarcoma, chondrosarcoma, osteogenic sarcoma, chordoma, angio sarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

### Pharmaceutical Compositions and Kits

An aspect of the disclosure provides a pharmaceutical composition comprising a therapeutically effective amount of an exosome, a therapeutically effective amount of an oHSV as described above and a pharmaceutically acceptable carrier. The pharmaceutical composition is useful for prophylaxis or treatment of a tumor in a subject. The pharmaceutical composition may be prepared in a suitable pharmaceutically acceptable carrier or excipient.

Another aspect of the disclosure provides a first pharmaceutical composition comprising a therapeutically effective amount of an exosome as described above and a pharmaceutically acceptable carrier. In addition, a second pharmaceutical composition is provided comprising a therapeutically effective amount of an oHSV as described above and a pharmaceutically acceptable carrier. In such aspect, a kit is provided to include the first pharmaceutical composition and the second pharmaceutical composition in a single package. The kit may further include a specification for use that a physician can refer during clinical use.

In some embodiments, the oHSV is a genetically engineered HSV-1 F strain expressing an immunostimulatory agent which is selected from GM-CSF, IL-2, IL-5, IL-12, IL-15, IL-24 and IL-27. In some embodiments, the oHSV is a genetically engineered HSV-1 F strain expressing IL-12 alone. In some embodiments, the oHSV is a genetically engineered HSV-1 F strain expressing both IL-12 and anti-PD-1 antibody.

Under ordinary conditions of storage and use, these preparations/compositions contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, sodium chloride or phosphate buffered saline. Prolonged absorption of the injectable compositions can be brought about by the use in the compositions of agents delaying absorption, for example, aluminum mono stearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### EXAMPLES

We describe the development of an adjunct therapy consisting of exosomes engineered to carry and release into tumor cells miRNAs specifically designed to target mRNAs encoding CTLA-4 checkpoint.

Exosomes are extracellular vesicles defined for the purposes of therapeutic applications by size and protein content. They package RNA and protein in cells in which they are produced and deliver the cargo to cells they are exposed. In the studies described in this report the desired exo some payload was a miRNA.

miRNAs are potent tools that in principle can be used to control the replication of certain protein coding RNAs. The objectives were to design miRNAs that can block the replication of cytotoxic T-lymphocyte-associated protein 4 and which could be delivered to infected cells via exosomes. We designed 10 miRNAs targeting the mRNA encoding CTLA4. Of the 10 miRNAs, miR-CTLA4-1#, miR-CTLA4-2#, miR-CTLA4-3# and miR-CTLA4-6# effectively blocked CTLA4 accumulation on transfection into susceptible cells. To facilitate packaging of the miRNA into exosomes we incorporated into the sequence of miR-CTLA4-3# an exosome packaging motif. As in our previous study, miR-CTLA4-3# could be packaged into exosomes and successfully delivered by exosomes to susceptible cells where it remained stable for at least 72 hrs. Moreover, miR-CTLA4-3# delivered to tumors via exosomes effectively reduced the expression of CTLA4.

Incorporation of RNAs into exosomes is sequence dependent and facilitated by hnRNPA2B1, a component of exosomes. hnRNPA2B1 sorts into exosomes RNAs containing one of two known exo some-packaging motifs (EXO-motifs). A key function of hnRNPA2B1 is to regulate mRNA trafficking to axons in neural cells that is mediated by binding a 21-nt RNA sequence called RNA trafficking sequence (RTS). This sequence contains both of the EXO-motifs.

We identified a murine tumor relatively resistant to the oncolytic activity of murine T1, T2 and T3 series of oHSV. The T1 series of oHSV is a HSV-1 F strain that does not express an immunoregulatory agent (also referred to as “T1012G” hereinafter). The T2 series of oHSV is a HSV-1 F strain that expresses IL-12 alone (also referred to as “T2850” hereinafter). The T3 series of oHSV is a HSV-1 F strain that simultaneously expresses IL-12 and anti-PD-1 antibodies (also referred to as “T3855” hereinafter).

The Examples show that the anti-tumor efficacy of T2850 and especially T3855 can be enhanced and the volume of tumors can be reduced by delivering to the tumors via the composition comprising T3855 (or T2850) and exosomes carrying a miRNA designed to target mRNA encoding CTLA-4.

### Materials and Methods

Syngeneic mouse model. The syngeneic mice were Balb/c for MFC tumor.

Plasmids expressing miR-CTLA-4. Target miRNA sequences against mouse CTLA4 were designed using Life Technologies' BLOCK-iT™ RNAi Designer and synthesized by Ige Biotechnology (Guangzhou, China). The synthesized miRNA fragments were digested with BamHI and XhoI restriction enzymes and cloned into the corresponding sites of pcDNA6.2-GW/EmGFP-miR-neg control plasmid (Invitrogen). The sequences of miRNAs are as follows:

miR-CTLA4-1#:
(SEQ ID NO. 53)
5′-AACCTTCAGTGGAGTTGGCGAGTTTTGGC
CACTGACTGACTcGCCAACCACTGAAGGTT-3′;
miR-CTLA4-2#:
(SEQ ID NO. 54)
5′-CTTCAGTGGAGTTGGCGAGCAGTTTTGGC
CACTGACTGACTGCTCGCCCtCCACTGAAG-3′;
miR-CTLA4-3#:
(SEQ ID NO. 55)
5′-ACTGTGCTGCGGAGGACAAATGTTTTGGC
CACTGACTGACATTTGTCCCGCAGCACAGT-3′;
miR-CTLA4-4#:
(SEQ ID NO. 56)
5′-CGACATTCACGGAGGAGAATAGTTTTGGC
CACTGACTGACTATTCTCCCGTGAATGTCG-3′;
miR-CTLA4-5#:
(SEQ ID NO. 57)
5′-GAACCTCACCCTCCAAGGACTGTTTTGGC
CACTGACTGACAGTCCTTGGGGTGAGGTTC-3′;
miR-CTLA4-6#:
(SEQ ID NO. 58)
5′-ATCCAAGGACTGGGAGCTGTTGTTTTGGC
CACTGACTGACAACAGCTCAGTCCTTGGAT-3′;
miR-CTLA4-7#:
(SEQ ID NO. 59)
5′-ACTCATGTACCCTCCGCCATAGTTTTGGC
CACTGACTGACTATGGCGGGGTACATGAGT-3′;
miR-CTLA4-8#:
(SEQ ID NO. 60)
5′-GGCAACGGGAGGCGGAGTTATGTTTTGGC
CACTGACTGACATAACTCCCTCCCGTTGCC-3′;
miR-CTLA4-9#:
(SEQ ID NO. 61)
5′-GGGCAACGGGACGGAGATTTAGTTTTGGC
CACTGACTGACTAAATCTCTCCCGTTGCCC-3′;
miR-CTLA4-10#:
(SEQ ID NO. 62)
5′-TGACTGTGCTGCGGCGGACAAGTTTTGGC
CACTGACTGACTTGTCCGCCAGCACAGTCA-3′;

The underline indicates the mature miRNA sequence.

The His-tagged mouse CTLA4 expression plasmid (mCTLA4-his) was purchased from YouBio Biotechnology (Changsha, China).

Cell lines. HEp-2 cells were obtained from the American Type Culture Collection and routinely cultured in DMEM (Life Technologies) supplemented with 5% (vol/vol) fetal bovine serum (FBS). MFC (Murine Forestomach Carcinoma) cells were kindly provided by JOINN Laboratories, Inc. (Beijing, China). B16 (Murine Melanoma) were kindly provided by Shenzhen International Institute for Biomedical Research (Shenzhen, China).

Antibodies. Antibodies used in this study were anti-His-tag (Cat No. 66005-1-Ig, Proteintech Group) and anti-GAPDH (Cat No. #2118, Cell Signaling Technology).

Exosome isolation. HEp-2 cells (5×10⁶) were transfected with 10 μg of plasmids expressing miR-CTLA-4. After 4 h incubation the cells were rinsed three times with PBS to exclude potential contamination of exosome in serum, and the cells were cultured in serum free medium for another 48 h. The supernatant fluid was harvested mixed with recommended dose of Total Exosome Isolation kit reagent (Thermo Fisher Cat No. 4478359), stored overnight at 4° C. and then centrifuged for 1 h. The pelleted exosomes were then resuspended in 200 μl of PBS or were lysed in RIPA buffer and then quantified by a BCA assay using the Enhanced BCA Protein Assay Kit (Beyotime Biotechnology, China) according to manufacturer's instructions. Exosome protein content was determined by calibration against standard curve, which was prepared by plotting the absorbance at 562 nm versus bovine serum albumin standard concentration.

Analyses of exosome size and quantifications. Exosome size distribution analysis was done using the qNano system (Izon, Christchurch, New Zealand). Izon's qNano technology (www.izon.com) was employed to detect extracellular vesicles passing through a nanopore by way of a single-molecule electrophoresis. In practice it enables accurate particle-by-particle characterization of vesicles from 75 to 150 nm in size of exosomes, without averaging the particle sizes. Purified exosomes were diluted to 1:10 in PBS with 0.05% Tween-20, vigorously shaken, and measured by using an NP150 (A45540) nanopore aperture according to the manufacturer's instructions. Data processing and analysis were carried out on the Izon Control Suite software v3.3 (Izon Science).

Immunoblot assays. Detection of His-tagged CTLA-4, GAPDH and exosome maker proteins by immunoblot assay. Cells were harvested and lysed with a RIPA lysis buffer (Beyotime) supplemented with 1 mM protease inhibitor phenylmethylsulfonyl fluoride (PMSF) (Beyotime) and phosphatase inhibitor (Beyotime). Cell lysates were heat denatured, and separated by SDS-PAGE, transferred to polyvinylidene difluoride membranes (Millipore). The proteins were detected by incubation with appropriate primary antibody, followed by horseradish peroxidase-conjugated secondary antibody (Pierce) and the ECL reagent (Pierce) and exposed to a film or images were captured using a ChemiDoc Touch Imaging System (Bio-Rad) and processed using ImageLab software. The densities of corresponding bands were quantified using ImageJ software.

oHSV construction. The construct of an exemplary oHSV (such as T2850 and T3855) involves a recombinant oncolytic Herpes Simplex Virus type 1 (HSV-1) comprising (a) a modified HSV-1 genome wherein the modification comprises a deletion between the promoter of U,56 gene and the promoter of Us1 gene of a wild-type HSV-1 genome such that (i) one copy of all double-copy genes is absent and (ii) sequences required for expression of all existing open reading frames (ORFs) in the viral DNA after the deletion are intact: and (b) a heterologous nucleic acid sequence encoding an immunostimulatory and/or immunotherapeutic agent, wherein the heterologous nucleic acid sequence is stably incorporated into at least the deleted region of the modified HSV-1 genome. Where only one heterologous nucleic acid sequence encoding an immunostimulatory or immunotherapeutic agent is inserted, the heterologous nucleic acid sequence is preferably incorporated into the deleted region of the genome. Where more than one heterologous nucleic acid sequences encoding immunostimulatory and/or immunotherapeutic agents are incorporated, a first heterologous nucleic acid sequences is preferably inserted into the deleted region of the genome. A second or further heterologous nucleic acid sequences may be inserted into the L component of the genome. A more detailed description of the construction and properties of the oncolytic herpes simplex virus (oHSV) is available from WO2017/181420.

### Results

Design and Production of Exosomes Containing miR-CTLA-4 (miR-CTLA-4 Exo).

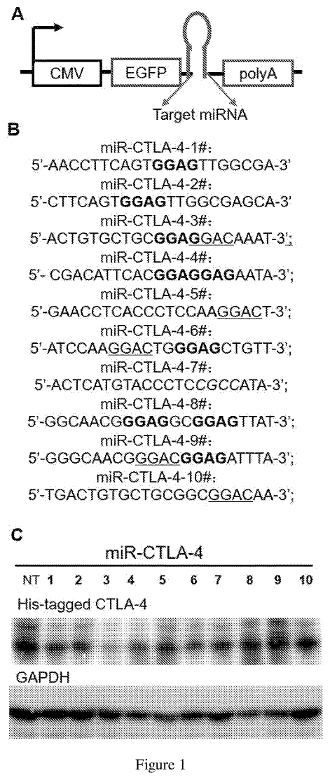

The objective of the first series of experiments was to design and product exosomes containing a miRNA targeting CTLA4. To this end we have first constructed 10 miRNAs designated miR-CTLA4-1#, miR-CTLA4-2#, miR-CTLA4-3#, miR-CTLA4-4#, miR-CTLA4-5#, miR-CTLA4-6#, miR-CTLA4-7#, miR-CTLA4-8#, miR-CTLA4-9# and miR-CTLA4-10#. The sequence of each of the miRNAs shown in FIG. 1 contains downstream of miRNA seed sequence additional sequences embodying exosome-packaging-associated motifs (exo-motifs). As illustrated in FIG. 1A, the miRNAs were cloned downstream of an open reading frame encoding EGFP into a miRNA expression vector named “pcDNA6.2-GW/EmGFP-miR-neg control plasmid” as described in Materials and Methods.

Then, to test the miRNAs HEp-2 cells were co-transfected with the miRNA expression vectors (miRmCTLA4-1#, miRmCTLA4-2#, miRmCTLA4-3#, miRmCTLA4-4#, miRmCTLA4-5#, miRmCTLA4-6#, miRmCTLA4-7#, miRmCTLA4-8#, miRmCTLA4-9#, miRmCTLA4-10#) described above and a plasmid encoding CTLA4 tagged at the C terminus with His (mCTLA4-His). As shown in FIG. 1B, miR-CTLA4-3# was the most effective of the 10 constructs in suppressing the accumulation of CTLA4. The results show that the accumulation of CTLA4 is repressed by miR-CTLA4-3# at higher efficiency. miR-CTLA4-1#, miR-CTLA4-2#, miR-CTLA4-4#, miR-CTLA4-5#, miR-CTLA4-6# and miR-CTLA4-7# showed moderate effect, whereas the non-targeting (NT), miR-CTLA4-8#, miR-CTLA4-9# and miR-CTLA4-10# plasmids had no effect on accumulation of CTLA4 (FIG. 1B). Therefore, miR-CTLA4-3# was selected for further studies.

In the next step we constructed exosomes encoding the selected miR-CTLA-4. HEp-2 cells seeded in T150 flask were transfected with 10 μg of the plasmid encoding the miR-CTLA4-3# or plasmid expresses non-target miRNA (NT). After 48 h the extracellular medium was harvested and the exosomes were purified as described in Materials and Methods.

Characterization of Exosome Carrying miR-CTLA-4.

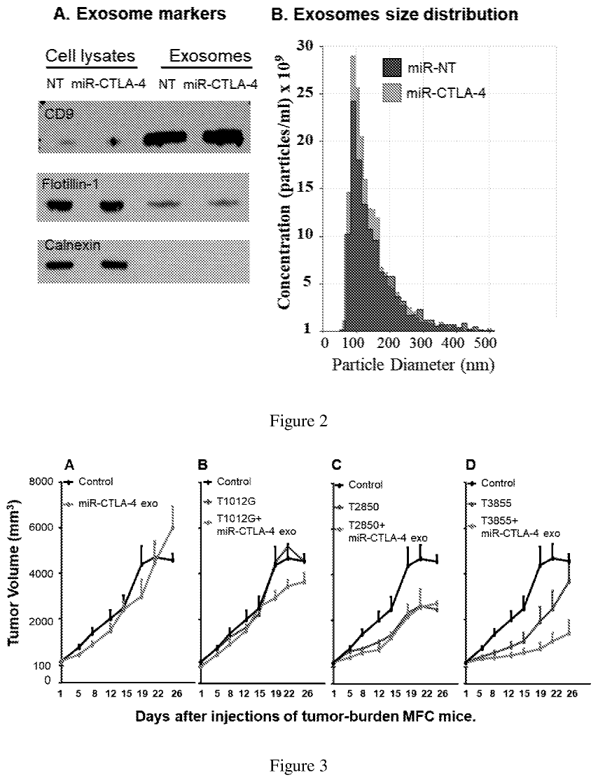

Several experiments were carried out to character the exosome carrying miR-CTLA-4. First, equal amounts of cells in which the exosomes were produced and equal amounts of exosomes were solubilized, subjected to electrophoresis in a denaturing gel were probed with antibodies to CD9, Flotilin-1 and Calnexin. As expected the result (FIG. 2A) shows that the exosomes contain CD9 and and Flotilin-1 but not Calnexin.

Next, the exosomes purified from HEp-2 cells transfected with 10 μg of the plasmid encoding the miR-CTLA4 or plasmid expresses non-target miRNA (miR-NT) measured with respect to size by nanoparticle tracking analysis using Izon's qNano technology. The result (FIG. 2B) shows that the exosomes produced by transfected cells average 100-200 nm in diameter.

Concurrent Administration of Exosomes Carrying miR-CTLA-4 and oHSV into MFC Implanted Tumors Enhances the Oncolytic Activity of T3 but not T1 or T2 oHSVs.

In the first step of this series of experiments, replicate cultures of HEp-2 were transfected with the plasmid of miR-CTLA4-3#, and the cells were cultured for 48 h. Then, the exo some produced in the HEp-2 (miR-CTLA-4 exo) were purified as described in Materials and Methods.

In the second step, oHSV T1012G, T2850 and T3855 were constructed as described in Materials and Methods. Then mouse forestomach carcinoma (MFC) cells were injected subcutaneously in the right flanks of 8 groups of 8 C57BL/6J mice for generating tumors. When the tumors reached an average of 80 mm³, they were intratumoral single injected with 1×10⁷ pfu of T1012G (Panel B), T2850 (Panel C) or T3855 (panel D) alone or in combination with 10 g of miR-CTLA-4 exosomes.

Finally, Tumor volumes were measured every 3 or 4 days until 26 days after injection. The result shows that the tumor volume in every group increased gradually after injection. FIG. 3A shows that the tumor volume of the mouse injected with miRNA-CTLA4 exo increased almost as fast as the tumor volume of the control, and he tumor volume of the mouse injected with miRNA-CTLA4 exo was larger than that of the control tumor at 26 days after injection. FIG. 3B shows that the tumor volume of control, T1012G and T1012G+miRNA-CTLA4 exo increased gradually after injection. After 26 days of injection, the tumor volume of the mouse injected with T1012G+miRNA-CTLA4 exo was smaller than that of the Control and T1012G. In FIG. 3C, the tumor volume of T2850 and T2850+miRNA-CTLA4 exo increased more slowly than that of the Control. After 26 days of injection, the tumor volume of T2850 and T2850+miRNA-CTLA4 exo were smaller than that of the Control. And the tumor volume of T2850+miRNA-CTLA4 exo did not differ significantly from the tumor volume of T2850 alone. In FIG. 3D, the tumor volume of T3855+miRNA-CTLA4 exo increased the slowest, followed by T3855, and finally the Control. After 26 days of injection, the tumor volume of the control was the largest, followed by the tumor volume of T3855, and the tumor volume of T3855+miRNA-CTLA4 exo was the smallest.

The results of this series of experiments shows that T2850 and T3855 can effectively inhibit the growth of tumors, and concurrent intratumoral administration of T3855 and miRNA-CTLA4 exo enhances the anti-tumor efficacy of T3855 and inhibits the growth of tumors.

The results show that miR-CTLA4-3# targets CTLA-4 and down-regulates the expression of CTLA-4. We have also shown that the miR-CTLA4-3# is packaged in exosomes and the purified exosomes contained CD9, Flotilin-1 but lacked Calnexin. The exosomes produced by Hep-2 Cells transfected with plasmid miR-CTLA4-3# or non-target miRNA (NT) average 100-200 nm in diameter. The results presented herein also show that an oHSV expressing IL-12 alone or both IL-12 and anti-PD-1 antibody can effectively inhibit the growth of tumors. Lastly, we have shown that concurrent intratumoral administration of the exosome carrying a miRNA targeting CTLA-4 and the oHSV expressing both IL-12 and anti-PD-1 antibody enhances the anti-tumor efficacy of the oHSV and inhibits the growth of tumors.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

All publications, patent applications, patents, and other references mentioned herein are expressly incorporated by reference in their entirety, to the same extent as if each were incorporated by reference individually. In case of conflict, the present specification, including definitions, will control. The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. A pharmaceutical composition or a kit for treating a tumor in a subject, comprising,
(a) a therapeutically effective amount of an exosome,
(b) a therapeutically effective amount of an oncolytic herpes simplex virus, and
(c1) a pharmaceutically acceptable carrier, for the pharmaceutical composition, or (c2) optionally instructions for use, for the kit,
wherein the exosome comprises an inhibitory amount of CTLA-4-targeting miRNA and an exo-motif operably linked to a seed sequence of the CTLA-4-targeting miRNA to enhance the packaging of the CTLA-4-targeting miRNA into the exosome, and
wherein the oncolytic herpes simplex virus expresses an immunostimulatory agent or both an immunostimulatory agent and an anti-PD-1 antibody.

2. The composition or the kit of claim 1, wherein the seed sequence of the CTLA4-targeting miRNA contains any one of the nucleic acid sequences of SEQ ID NO. 1 to SEQ ID NO. 4.

3. The composition or the kit of claim 1, wherein the exo-motif is selected from a group consisting of nucleic acid sequence of SEQ ID NO. 21 to SEQ ID NO. 49.

4. The composition or the kit of claim 1, wherein the exo-motif is located downstream and linked to the seed sequence of the CTLA4-targeting miRNA covalently.

5. The composition or the kit of claim 1, wherein the exo-motif is obtained by mutation of one or more nucleic acids of the CTLA4 targeting miRNA except for the seed sequence.

6. The composition or the kit of claim 1, wherein the exo-motif is a two-fold motif generated through combination of two single exo-motifs, wherein any of the two single exo-motifs is selected from a group consisting of nucleic acid sequence of SEQ ID NO. 21 to SEQ ID NO. 47.

7. The composition or the kit of claim 6, wherein the two-fold motif has a nucleic acid sequence of SEQ ID NO. 48.

8. The composition or the kit of claim 1, wherein CTLA4-targeting miRNA and the exo-motif, when operably linked, share at least one nucleotide or two nucleotides or connect through a linker.

9. The composition or the kit of claim 8, wherein the linker consists of two or more nucleotides selected from a group consisting of Adenine (A), Guanine (G), Cytosine (C), Thymine (T) and Uracil (U).

10. The composition or the kit of claim 9, wherein the linker is -GC-.

11. The composition of claim 1, wherein the CTLA4-targeting miRNA and the exo-motif, when operably linked, has a nucleic acid sequence of SEQ ID NO. 7.

12. The composition or the kit of claim 1, wherein the immunostimulatory agent is selected from GM-CSF, IL-2, IL-5, IL-12, IL-15, IL-24 and IL-27.

13. The composition or the kit of claim 12, wherein the immunostimulatory agent is IL-12.

14. (canceled)

15. The composition or the kit of claim 1, wherein the oncolytic herpes simplex virus expresses both IL-12 and an anti-PD-1 antibody.

16. The composition or the kit of claim 1, wherein the oncolytic herpes simplex virus is an HSV-1 expressing IL-12 and anti-PD-1 antibody.

17. The composition or the kit of claim 16, wherein the HSV-1 is F strain of an HSV-1.

18. The composition or the kit of claim 17, wherein a fragment of nucleotide sequence from 117005 to 132096 of native backbone is deleted.

19. The composition or the kit of claim 1, wherein the tumor is a malignant tumor.

20. The composition or the kit of claim 19, wherein the malignant tumor is selected from a group consisting of melanoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, gastric carcinoma and forestomach carcinoma.

21. The composition or the kit of claim 1, wherein the subject is human.

22-42. (canceled)

43. A method for treating a tumor in a subject, comprising administering to the subject the pharmaceutical composition or the kit of claim 1.
